SEQUENCE LISTING (1 sequence)

SEQ ID NO: 1 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

(19) United States
(12) Patent Application Publication
(10) Pub. No.: US 2023/0255986 A1
(43) Pub. Date: Aug. 17, 2023

(54) PHARMACEUTICAL COMPOSITIONS FOR TREATING INFECTIONS BY A NEUROTROPIC VIRUS

(71) Applicants: CERES BRAIN Therapeutics, Paris (FR); Commissariat à l`Energie Atomique et aux Energies Alternatives (CEA), Paris (FR)

(72) Inventors: Henri BENECH, Paris (FR); Aloïse MABONDZO, Paris (FR); Thomas JOUDINAUD, Paris (FR)

(21) Appl. No.: 18/013,191

(22) PCT Filed: Jul. 15, 2021
(86) PCT No.: PCT/EP2021/069761

§ 371 Date: Dec. 27, 2022

(30) Foreign Application Priority Data
Jul. 15, 2020 (EP) 20305807.8

(51) Int. Cl. A61K 31/675; A61K 9/00; A61K 9/107; A61P 31/18; A61K 31/4706; A61K 31/706

(52) U.S. Cl. CPC A61K 31/675; A61K 9/0043; A61K 9/1075; A61P 31/18; A61K 31/4706; A61K 31/706

(57) ABSTRACT

The present invention relates to a pharmaceutical composition, comprising at least one antiviral agent as an active agent, for use in the prevention or treatment of an infection by a neurotropic virus, in particular by a virus of the Coronaviridae family or by a virus of the Retroviridae family, wherein the pharmaceutical composition is administered by the nasal route, in particular through the olfactory nerve pathway.

### FIELD OF THE INVENTION

The present invention relates to compositions and methods for treating infections by neurotropic viruses, in particular by viruses of the Coronaviridae family or by viruses of the Retroviridae family, more particularly by the virus SARS-CoV-2 or by the human immunodeficiency virus (HIV).

### TECHNICAL BACKGROUND

In December 2019 an outbreak of pneumonia cases of unknown origin in occurred in Wuhan in China and spread quickly nationwide. On Jan. 7, 2020, the causative pathogen was identified as a novel coronavirus, which was named 2019-nCoV and later SARS-CoV-2.

The new virus is closely related to both SARS-CoV (82% nucleotide identity) and MERS-CoV (50% nucleotide identity), yet distinct from them.

Early mortality rates suggested that COVID-19, the name for the disease caused by SARS-CoV-2, may be less severe than SARS and MERS. However, illness onset among rapidly increasing numbers of people rapidly suggested that SARS-CoV-2 would be more contagious than both SARS-CoV and MERS-CoV. As of May 11, 2020 4 063 525 cases of COVID-19 (in accordance with the applied case definitions and testing strategies in the affected countries) have been reported, including 282 244 deaths. Several scientific articles reported neurologic disorders in patients infected by SARS-CoV-2. The mechanism is still unclear and could be either caused by the virus penetrating the brain via the olfactory nerves or caused by an inflammatory process reaching the brain. Nevertheless, others virus are able to reach the brain and stay for months, years or decades.

A great deal of efforts has been made to find effective drugs against the SARS-CoV-2. Among the various compounds tested, remdesivir, a drug previously developed for the treatment of Ebola virus infections, has been reported to show promising efficacy and acceptable safety in treating COVID-19 in a news release of the National Institutes of Health (NIH) dated Apr. 29, 2020. As such, preliminary results indicate that patients who received remdesivir had a 31% faster time to recovery than those who received placebo (p<0.001). Specifically, the median time to recovery was 11 days for patients treated with remdesivir compared with 15 days for those who received placebo. Results also suggested a survival benefit, with a mortality rate of 8.0% for the group receiving remdesivir versus 11.6% for the placebo group (p=0.059).

However, the efficacy of remdesivir is not completely established yet as Wang et al. (2020) Lancet doi.org/10.1016/S0140-6736(20)31022-9 report that in their trial remdesivir use was not associated with a difference in time to clinical improvement.

Accordingly, there is still a need for alternative treatments of infections by SARS-CoV-2, in particular to prevent or treat its neurological effects.

More generally, a large number of viruses, among which Retroviridae viruses, are able to infect central nervous system (CNS), in particular the brain, of infected subject and to produce neurologic disorders, such as HIV-associated neurocognitive disorders (HAND), which intensity ranges from mild to dementia.

These virus-caused neurological disorders are also in need of alternative treatments.

### SUMMARY OF THE INVENTION

The present invention follows from the unexpected finding, by the inventors, that administration of antiviral agents through the olfactory nerve route could improve their efficacy against neurotropic viruses, in particular against viruses of the Coronaviridae family or by viruses of the Retroviridae family, more particularly against SARS-CoV-2 or the human immunodeficiency virus (HIV).

Without wishing to be bound to a particular theory, the inventors believe that one of the reasons antiviral drugs used against SARS-CoV-2 or HIV are ineffective lies in the fact that the virus could escape these drugs by circulating in the nervous system where the drugs cannot reach them, in particular through infection of the olfactory nerve.

Accordingly, the present invention relates to a pharmaceutical composition, comprising at least one antiviral agent as an active agent, for use in the prevention or treatment of an infection by a neurotropic virus in an individual, in particular by a virus of the Coronaviridae family or of the Retroviridae family, wherein the pharmaceutical composition is administered by the nasal route, in particular through the olfactory nerve pathway.

The present invention also relates to a method for preventing or treating an infection by a neurotropic virus in an individual, in particular by a virus of the Coronaviridae family or of the Retroviridae family, comprising administering to the individual a pharmaceutical composition comprising at least one antiviral agent as an active agent, wherein the pharmaceutical composition is administered by the nasal route, in particular through the olfactory nerve pathway.

### DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

### Virus

As intended herein the infection to be prevented or treated is due to a neurotropic virus. Neurotropic viruses, which are well known to one of skill in the art, are capable of infecting nerve cells. Neurotropic viruses according to the invention can be neuroinvasive and/or neurovirulent. Neurotropic viruses according to the invention can infect the central nervous system, in particular the brain, and/or the peripheral nervous system.

Neurotropic viruses according to the invention are preferably selected from the group consisting of Japanese Encephalitis virus, Venezuelan Equine Encephalitis virus, California encephalitis virus; polio virus, coxsackie virus, echo virus, mumps virus, measles virus, influenza virus, rabies virus, varicella-zoster virus, Epstein-Barr virus, cytomegalovirus, HHV-6 virus, rubella virus, JC virus, a virus of the Retroviridae family, a virus of the Herpesviridae family, a virus of the Coronaviridae family and mutants and variants thereof.

A preferred neurotropic virus of the Retroviridae family according to the invention is Human Immunodeficiency Virus (HIV), in particular HIV-1 or HIV-2, human T-lymphotropic virus 1, or mutants or variants thereof.

A preferred neurotropic virus of the Herpesviridae family according to the invention is Herpes Simplex Virus (HSV), in particular HSV-1 or HSV-2, or mutants or variants thereof. Preferably, the infection to be prevented or treated is due to a virus of the Coronaviridae family.

Preferably, the virus as defined above is of the Alphacoronavirus, Betacoronavirus, Deltacoronavirus, or Gammacoronavirus genus, more preferably of the Betacoronavirus genus, most preferably of the Sarbecovirus or the Merbecovirus sub-genus.

Preferably also the virus as defined above is a human virus, i.e. a virus which can infect a human.

Preferably, the virus as defined above is selected from the group consisting of SARS-CoV, SARS-CoV-2, MERS-CoV and mutants or variants thereof.

Preferably, the virus as defined above is SARS-CoV-2, or a mutant or variant thereof. SARS-CoV-2 is notably described in Fuk-Woo Chan et al. (2020) Emerging Microbes & Infections 9:221-236, which is incorporated herein by reference, and is also named 2019-nCoV, HCoV-19, SARS2, COVID-19 virus, Wuhan coronavirus, Wuhan seafood market pneumonia virus, Human coronavirus 2019.

Preferably, the virus as defined above is SARS-CoV-2 and has the genomic sequence defined by NCBI Reference Sequence NC_045512.2 (SEQ ID NO: 1), or the complementary thereof, or is a mutant or variant thereof. Mutant or variants of SEQ ID NO: 1 can in particular be found on the “NCBI virus” website by searching for SARS-CoV-2 taxid:2697049. A preferred variant of SARS-CoV-2 according to the invention harbours at least one mutation, in particular of the spike protein, selected from the group consisting of K417N, K417T, L452R, T478K, E484K, E484Q, N501Y and D614G. A preferred variant of SARS-CoV-2 according to the invention is a variant of concern, more preferably selected from the group consisting in B.1.1.7 (Alpha), B.1.1.7+E484K, B.1.351 (Beta), P.1 (Gamma), B.1.617.1 (Kappa), B.1.617.2 (Delta) and B.1.617.3.

As intended herein, a “mutant or variant” of a virus as defined above, or of a genomic sequence of a virus as defined above, has a genomic sequence or is a nucleotide sequence which has at least 85%, 90%, 95%, 96% 97%, 98%, 99% or 99.5% identity with the genomic sequence of the virus as defined above.

As intended herein, a first nucleotide sequence “having at least X % identity” with a second nucleotide sequence, in particular differs from the second sequence by the insertion, the suppression or the substitution of at least one nucleotide. Besides, the percentage of identity between two nucleotide sequences is defined herein as the number of positions for which the bases are identical when the two sequences are optimally aligned, divided by the total number of bases of the longer of the two sequences. Two sequences are said to be optimally aligned when the percentage of identity is maximal. Besides, as will be clear to one of skill in the art, it may be necessary to add gaps in order to obtain an optimal alignment between the two sequences. In addition, when calculating the percentage of identity between an RNA nucleotide sequence and a DNA nucleotide sequence, an Uracile (U) base and a Thymine (T) base at the same position are considered to be identical.

As intended herein preventing or treating an infection by a neurotropic virus in an individual, encompasses preventing or treating the symptoms, disorders, syndromes, conditions or diseases associated to the infection by the neurotropic virus.

As intended herein preventing or treating an infection by a virus of the Coronaviridae family in an individual, encompasses preventing or treating the symptoms, disorders, syndromes, conditions or diseases, such as pneumonia or COVID-19, associated to the infection by the virus of the Coronaviridae family, more particularly by SARS-CoV-2.

Preferably, the pharmaceutical composition as defined above is for use for preventing or treating anosmia associated with the viral infection.

Preferably, the pharmaceutical composition as defined above is for use for preventing or treating dysgeusia associated with the viral infection.

Preferably, the pharmaceutical composition as defined above is for use for preventing or treating encephalopathy associated with the viral infection.

Preferably, the pharmaceutical composition as defined above is for use for preventing or treating neuro-inflammation associated with the viral infection.

Preferably, the pharmaceutical composition as defined above is for use for preventing or treating a neurological disorder associated with the viral infection.

Preferably, the pharmaceutical composition as defined above is for use for preventing or treating a neurocognitive disorder associated with the viral infection, in particular for preventing or treating HIV-associated neurocognitive disorder (HAND).

### Individual

Preferably, the individual is a bird, such as a chicken, or a mammal, such as a human, a canine, in particular a dog, a feline, in particular a cat, an equine, a bovine, a porcine, a caprine, such a sheep or a goat, or a camelidae, more preferably the individual is a human.

The individual as defined above may be of any age. Preferably, the individual as defined above is a human aged 20 or more, 30 or more, 40 or more or 50 or more, more preferably 60 or more, even more preferably 70 or more and most preferably 80 or more. Preferably, the individual as defined above is a human aged less than 80, 70, 60, 50, 40, 30 or 20.

The individual as defined above may be a male or a female. Preferably, the individual as defined above is a male individual.

Preferably, the individual as defined above suffers from at least one other disease or condition, in particular selected from hypertension, diabetes, in particular type 2 diabetes, metabolic syndrome, a cardiovascular disease, in particular ischemic cardiomyopathy, a chronic respiratory disease, or cancer.

Preferably, the individual as defined above is overweight or obese.

According to a usual definition a human individual is considered overweight if its Body Mass Index (BMI, body weight in kg relative to the square of the height in meters) is higher than or equal to 25 kg/m² and less than 30 kg/m² and the individual will be said obese if his BMI is higher than or equal to 30 kg/m². The individual according to the invention may notably present with severe obesity, in particular characterized in human by a BMI higher than or equal to 35 kg/m².

More generally, it is preferred that the individual as defined above is a human and has a BMI higher than or equal to 25 kg/m², 26 kg/m², 27 kg/m², 28 kg/m², 29 kg/m², 30 kg/m², 31 kg/m², 32 kg/m², 33 kg/m², 34 kg/m², 35 kg/m² or 40 kg/m².

Besides, the individual as defined above may also have an abdominal obesity, corresponding in particular to a visceral adipose tissue excess. According to a usual definition a male human individual has an abdominal obesity if the abdominal perimeter is higher than or equal to 94 cm, in particular higher than 102 cm and a female human individual has an abdominal obesity if the abdominal perimeter is higher than or equal to 80 cm, in particular higher than 88 cm. The abdominal perimeter measure is well known to one of skilled in the art: abdomen circumference is thus preferably measured midway between the last floating rib and the top of the iliac crest in a standing individual in gentle expiration.

It is particularly preferred that the individual as defined above is a man and presents with an abdominal perimeter higher than or equal to 90 cm, 91 cm, 92 cm, 93 cm, 94 cm, 95 cm, 96 cm, 97 cm, 98 cm, 99 cm, 100 cm, 101 cm or 102 cm. It is also preferred that the individual according to the invention is a woman and presents with an abdominal perimeter higher than or equal to 75 cm, 76 cm, 77 cm, 78 cm, 79 cm, 80 cm, 81 cm, 82 cm, 83 cm, 84 cm, 85 cm, 86 cm, 87 cm or 88 cm.

Preferably, the individual according to the invention is afflicted with Acquired Immunodeficiency Syndrome (AIDS) or is at risk of being afflicted with AIDS.

Preferably, the individual according to the invention is afflicted with COVID-19 or is at risk of being afflicted with COVID-19.

Preferably, the individual according to the invention is afflicted with anosmia or is at risk of being afflicted with anosmia.

Preferably, the individual according to the invention is afflicted with dysgeusia or is at risk of being afflicted with dysgeusia.

Preferably, the individual according to the invention is afflicted with encephalopathy or is at risk of being afflicted with encephalopathy.

Preferably, the individual according to the invention is afflicted with neuro-inflammation or is at risk of being afflicted with neuro-inflammation.

Preferably, the individual according to the invention is afflicted with a neurological disorder or is at risk of being afflicted by a neurological disorder.

Preferably, the individual according to the invention is afflicted with a neurocognitive disorder, in particular HIV-associated neurocognitive disorder (HAND) or is at risk of being afflicted with a neurocognitive disorder, in particular HIV-associated neurocognitive disorder (HAND).

### Antiviral Agents

As intended herein any antiviral agent which can prevent or treat an infection by a neurotropic virus, in particular by a virus of the Coronaviridae family or of the Retroviridae family, can be used according to the invention. The antiviral agent may in particular be an inhibitor of the viral polymerase (such as Nsp12 of SARS-CoV-2 or the reverse transcriptase (RT) of HIV), of the viral helicase (such as Nsp13 or SARS-CoV-2), of the viral integrase (such as HIV integrase) or of the viral protease (such HIV protease); an inhibitor of the binding of the virus to its target receptor(s) (such as ACE-2 for SARS-CoV2-2 or CD4, CCR5 or CXCR4 for HIV); a fusion inhibitor; or a general antiviral agent, such as an interferon.

Preferably, the antiviral agent according to the invention is a substrate of an efflux transporter of the individual. Efflux transporters, also named efflux cell membrane transporters, are well known to a person skilled in the art. Efflux transporters may be responsible for drug resistance, by transporting substrates drugs, which targets are intracellular, outside of the cell. Examples of efflux transporters according to the invention include P-glycoprotein (P-gp/ABCB1), multidrug resistance-associated protein 2 (MRP2/ABCC2), and breast cancer resistance protein (BCRP/ABCG2).

However it is preferred that the antiviral agent is selected from the group consisting of hydroxychloroquine; chloroquine; atovaquone; remdesivir; ribavirin; didanosine; penciclovir; favipravir; nafamostat; cobicistat; nitazoxanide; azithromycin; carrimycin; recombinant human interferon α1β; peginterferon alfa-2a; oseltamivir; nicotine; tofacitinib; imatinib; acalabrutinib; losartan; valsartan; telmisartan; tetrandine; clevudine; tiliquinone; nitazoxanide; tilbroquinone; nifurzide; nifuroxazide; minocycline; rovomycine; rocephine; HIV reverse transcriptase inhibitors, such as tenofovir; HIV protease inhibitors, such as lopinavir, ritonavir, lopinavir/ritonavir, darunavir, danoprevir, umifenovir; purine analogues; and pharmaceutically acceptable salts, derivatives and prodrugs thereof.

More preferably, the antiviral agent is selected from the group consisting of remdesivir, hydroxychloroquine, tenofovir and lopinavir. Most preferably, the antiviral agent is molnupiravir,

Preferably also, the antiviral agent is an anti-HIV agent, in particular selected from the group consisting in:
abacavir,
abacavir/lamivudine,
abacavir/lamivudine/zidovudine,
atazanavir,
atazanavir/cobicistat,
bictegravi/tenofoviral afenamide,
cabotegravir/rilpivirine,
darunavir,
darunavir/cobicistat,
darunavir/cobicistat/tenofovir alafenamide/emtricitabine,
delavirdine,
didanosine,
dolutegravir,
dolutegravir/abacavir/lamivudine,
dolutegravir/lamivudine,
dolutegravir/rilpivirine,
doravirine,
doravirine/tenofovir disoproxil fumarate/lamivudine,
efavirenz,
efavirenz/tenofovir disoproxil fumarate/emtricitabine,
elvitegravir,
elvitegravir/cobicistat/tenofovir disoproxil fumarate/emtricitabine,
elvitegravir/tenofovir alafenamide/emtricitabine,
emtricitabine,
emtricitabine/tenofovir alafenamide,
emtricitabine/tenofovir disoproxil fumarate,
enfuvirtide,
etravirine,
fosamprenavir,
fostemsavir
ibalizumab-uiyk,
indinavir,
lamivudine,
lopinavir ritonavir,
maraviroc,
nelfinavir,
nevirapine,
raltegravir,
rilpivirine,
rilpivirine/tenofovir disoproxil fumarate/emtricitabine,
rilpivirine/tenofovir alafenamide/emtricitabine,
ritonavir,
saquinavir,
stavudine,
tenofovir alafenamide,
tenofovir disoproxil fumarate,
tenofovir disoproxil fumarate/lamivudine,
tipranavir,
zidovudine, and
zidovudine/lamivudine.

Preferably, the antiviral agent as defined above has a log P value of at least −3; −2.5; −2; −1.5; −1; −0.5; 0; 0.5; 1; 1.5; 2; 2.5; 3; 3.5; 4; 4.5; 5; 5.5 or 6.

Preferably, the antiviral agent as defined above has a log P value below 6; 5.5; 5; 4.5; 4; 3.5; 3; 2.5; 2; 1.5; 1; 0.5; 0; −0.5; −1; −1.5; −2; −2.5; or −3.

The log P value gives an indication of the ability of an antiviral agent to diffuse across biological lipidic membranes. The higher the log P value, the higher the liposolubility of the antiviral agent. Antiviral agents with a log P value above 1, 2 or 3, preferably above 2, are generally considered to have an ability to diffuse across biological lipidic membranes. Determination of a log P (i.e. log₁₀ P) value is well known to the person skilled in the art. P, also referred to as K_{ow}, refers to the n-octanol-water partition coefficient or ratio. For a given substance P=(concentration of the substance in an octanol-rich phase)/(concentration of substance in a water-rich phase). The octanol-rich phase contains essentially only octanol and the water-rich phase contains essentially only water, e.g. distilled water. In case, the substance exists as several species the concentration of the substance is the sum of the concentrations of the several species.

Advantageously, where the antiviral agent according to the invention is a substrate of an efflux transporter, the composition according to the invention may compensate for the transportation of the antiviral agent outside of target cells by improving its penetration into target cells, even for an antiviral agent with a good ability to diffuse across biological lipidic membranes, i.e. such as an antiviral agent with a log P value above 1, 2 or 3. Accordingly, in an embodiment of the invention the antiviral agent is a substrate of an efflux transporter of the individual and has a log P value above 1, 2 or 3, preferably above 2.

Preferably, the antiviral agent as defined above has a water solubility of at least 0.001 mg/mL, 0.005 mg/mL, 0.01 mg/mL, 0.05 mg/mL, 0.1 mg/mL or 0.5 mg/mL.

Preferably, the antiviral agent as defined above has a water solubility of less than 0.5 mg/mL, 0.1 mg/mL, 0.05 mg/mL, 0.01 mg/mL, 0.005 mg/mL or 0.001 mg/mL.

Determination of water solubility of a substance is well known to the person skilled in the art. It is the saturation concentration (m/v) of the substance in substantially pure water, e.g. distilled water.

Preferably, the log P and water-solubility values mentioned herein are measured under normal conditions of temperature (20° C.) and pressure (1 atm), and at a pH of 7. By way of example:
Tenofovir disoproxil fumarate (TDF) has a log P value of about 1.25 and a water solubility of about 13.4 mg/ml (in distilled water at 25° C.);
Tenofovir alafenamide has a log P value of about 1.6 and a water solubility of about 4.86 mg/ml (at 20° C.);
Emtricitabine has a log P value of about −0.43 and a water solubility of about 112 mg/ml;
Dolutegravir has a log P value of about 2.2 and is slightly soluble;
Abacavir has a log P value of about 1.2 and a water solubility of about 77 mg/ml (for the sulfate salt);
Lamivudine has a log P value of about −1.4 and a water solubility of about 70 mg/ml;

Preferably, the antiviral agent according to the invention has a molecular mass below 10 000 g/mol, below 5 000 g/mol, below 2 500 g/mol or below 1 000 g/mol.

Preferably, the antiviral agent according to the invention is selected from the group consisting of tenofovir disoproxil, in particular tenofovir disoproxil fumarate (TDF), tenofovir alafenamide, in particular tenofovir alafenamide fumarate (TAF), emtricitabine, dolutegravir, abacavir, in particular abacavir sulfate, lamivudine, and combinations thereof.

In an embodiment of the invention, the antiviral agent according to the invention is not an interferon. In another embodiment of the invention, the antiviral agent according to the invention is not ribavirin.

### Pharmaceutical Composition

Preferably, the pharmaceutical composition as defined above further comprises at least one pharmaceutically acceptable vehicle, carrier or excipient suitable for an administration through the nasal route, in particular through the olfactory nerve pathway.

Vehicles, carriers or excipients suitable for an administration through the nasal route are well known to one of skill in the art and may in particular be selected from the group consisting of a fatty or a lipophilic solvent or phase and an aqueous or hydrophilic solvent or phase.

The fatty or lipophilic solvent or phase may comprise or consist of at least one fatty acid, monoglyceride, diglyceride, triglyceride, fatty alcohol, fatty ester or combinations thereof.

By way of example, the fatty acid may in particular be docosahexaenoic acid or isopropyl palmitate.

By way of example, the monoglyceride may in particular by a mixture of monoacylglycerols obtained by direct esterification of glycerol with carpylic (octanoic) and capric (decanoic) acids, such as CAPMUL® MCM NF (Abitec).

By way of example, the triglyceride may in particular be Caprylic/Capric Triglyceride. Maisine® CC (Gattefossé), also named Glycerol/Glyceryl monolinoleate, is an example of a combination of mono-, di-, and triglycerides.

By way of example, the fatty alcohol may in particular be myristat isopropyl alcohol or stearyl alcohol.

By way of example, the fatty ester may in particular be isopropyl lauroyl sarcosinate.

The aqueous or hydrophilic solvent or phase may comprise or consist of water, a glycol, such as glycerol, carboxymethyl cellulose, benzyl alcohol or combinations thereof.

The fatty or lipophilic solvent phase may also comprise or consist of 2-(2-ethoxyethoxy)ethanol, such as TRANSCUTOL® (Gattefossé).

The aqueous or hydrophilic solvent or phase may also comprise with pH-modifiers such as citric acid or hydrochloric acid.

The pH of the of the aqueous solvent or phase is preferably of from 5 to 7, more preferably of 5 to 6.

The aqueous an aqueous or hydrophilic solvent or phase may also comprise at least one salt such as citrate salts, anhydrous monosodium phosphate, disodium phosphate or sodium chloride.

Preferably, the pharmaceutical composition as defined above further comprises at least one tensio-active, emulsifier or co-surfactant agent, in particular selected from the group consisting of polysorbate 80, cocoglycoside, sugar esters, polyol esters, such as sorbitane esters (e.g. Span 80), lecithine, cholesterol, a glycol, polyethylene glycols and derivative thereof, polyglycerols and creatine dodecyl ester.

As is well known to a person of skill in the art, a glycol refers to an alkyl-diol. Preferred glycols according to the invention are ethylene glycol, i.e. ethane-1,2-diol; propylene glycol, i.e. propane-1,2-diol; propane-1,3-diol; butane-2,3-diol; or 2-methylpropane-1,3-diol.

Preferably, the tensio-active, emulsifier or co-surfactant agent represents no more than 15% w/w of the pharmaceutical composition.

At least one preservative agent, such as benzalkonium chloride, may also be comprised the pharmaceutical composition as defined above. The preservative agent, such as benzalkonium chloride, is preferably at a concentration of less than 0.1% (m/m). The preservative agent, such as benzalkonium chloride, is preferably at a concentration of more than 0.001% (m/m).

Preferably, the pharmaceutical composition as defined above is in the form of a formulation selected from the group consisting of a lipid solution, an oil-in-water micro-emulsion, an aqueous solution, a glycolic aqueous solution, a water-in-oil micro-emulsion, a micellar solution, and a reverse micellar solution.

Emulsions, in particular nano- or micro-emulsions, are well known to one of skill in the art and are notably described in Ullio-Gamboa et al. (2019) Nanomedicine 14:1579-1593, which is incorporated herein by reference. By way of example, for lipophilic antiviral agents, i.e. having a low water solubility and/or log P >3, such as hydroxychloroquine or lopinavir, the pharmaceutical composition can be formulated as either:
a lipid solution, or
an oil-in-water micro-emulsion having an aqueous (i.e. hydrophilic) phase and a fatty (i.e. lipophilic) phase in which the antiviral agent is solubilized.

At least one tensio-active, emulsifier or co-surfactant agent can be added to the micro-emulsion.

By way of example, for hydrophilic antiviral agents, i.e. having a high water solubility and/or log P <3, such as remdesivir or tenofovir, the pharmaceutical composition as defined above can be formulated as either:
an aqueous solution optionally comprising at least one pH-modifier, at least one salt, or at least one glycol, or
a water-in-oil micro-emulsion having an aqueous (i.e. hydrophilic) phase in which the antiviral agent is solubilized, and a fatty (i.e lipophilic) phase.

At least one tensio-active, emulsifier or co-surfactant agent can be added to the micro-emulsion.

Preferably, the pharmaceutical composition according to the invention comprises at least one antiviral agent having a log P value below 3, 2 or 1, more preferably below 2, and is formulated as a water-in-oil micro-emulsion.

Preferably, the water-in-oil micro-emulsion according to the invention comprises an oil (i.e. fatty) phase at 60-80% (mass of the oil phase/mass of the water-in-oil micro-emulsion) and a water (i.e. aqueous) phase at 20-40% (mass of the water phase/mass of the water-in-oil micro-emulsion).

More preferably, the water-in-oil micro-emulsion according to the invention comprises an oil (i.e. fatty) phase at 60-80% (m/m) which consists of (i) a mixture of monoglycerides and a glycol, in particular propylene glycol, at 50-60% (m/m), and (ii) at least one tensio-active, emulsifier or co-surfactant agent at 10-20% (m/m), and a water (i.e. aqueous) phase at 20-40% (m/m).

The mixture of monoglycerides and the glycol, in particular propylene glycol, may notably comprise 25-35% (m/m) of monoglycerides and 25-35% (m/m) of the glycol, in particular propylene glycol.

The at least one tensio-active, emulsifier or co-surfactant agent may be a mixture of Span 80, Polysorbate 80 and propylene glycol.

Optionally, the water-in-oil micro-emulsion according to the invention may further comprise a preservative, such as benzalkonium chloride,

### Administration

The pharmaceutical composition is administered by the nasal route, in particular through or by targeting the olfactory nerve pathway. To this end it is preferred that the composition is administered by the intranasal route, i.e. an intranasal, administration is preferred.

As intended herein nasal administration, in particular intranasal, administration, is such that the nasal mucosa of the nasal cavity is contacted by the pharmaceutical composition as defined above. More particularly, nasal administration, in particular intranasal, administration, is such that the nasal mucosa of the olfactory area of the nasal cavity is contacted by the pharmaceutical composition as defined above.

Numerous methods and devices for nasal, in particular intranasal, administrations are known to one of skill in the art. In particular, the pharmaceutical composition as defined above can be administered as a nasal lavage, as nasal drops, with a squirt system or with a sprayer.

The devices for nasal administration may be single-dose, bi-dose or multidose devices. Single-dose or bi-dose device are especially adapted for administering preservative-free pharmaceutical compositions.

### EXAMPLES

Examples of pharmaceutical compositions according to the invention are given below for the following compounds depending on their octanol-water ratio (Log P) and water solubility.

| | | | Water | | Molecular | |
|---|---|---|---|---|---|---|
| Compounds | LogP | pKa | solubility | | weight | |
| Tenofovir disoproxyl | 1.25 | 3.75 | 13.4 | mg/mL | 635.51 | g/mol |
| fumarate | | | | | | |
| Hydroxychloroquine | 3.87 | 15.59 | 0.0261 | mg/mL | 335 | g/mol |
| Remdesivir | 2.01 | 10.23 | 0.339 | mg/mL | 602.58 | g/mol |
| Lopinavir | 3.9 | 13.39 | 0.00192 | mg/mL | 628.80 | g/mol |

### Example 1: Pharmaceutical Composition Comprising Tenofovir Disoproxil Fumarate

The composition comprises a hot water-in-oil micro-emulsion incorporating tenofovir disoproxil fumarate at a concentration of from 1 to 100 mg/ml, wherein the oil phase comprises soybean oil/Span 80/Tween 80/Propylene glycol with respective amounts of 21/5/1/6 (in weight), with the addition of benzalkonium chloride (less than 0.1 w %).)

### Example 2: Pharmaceutical Composition Comprising Hydroxychloroquine

The composition comprises a micro-emulsion incorporating hydroxychloroquine at a concentration of from 1 to 20 mg/ml, wherein the micro-emulsion is made of MAISINE®/Span 80/water with respective amounts of 75 to 96 w %/2 to 15 w %/2 to 10 w %, with the addition of benzalkonium chloride (less than 0.1 w %).

### Example 3: Pharmaceutical Composition Comprising Remdesivir

The composition comprises a micro-emulsion incorporating remdesivir at a concentration of from 1 to 100 mg/ml, wherein the micro-emulsion is made of propylene glycol/glycerine/water with respective amounts of 2 to 8 w %/2 to 8 w %/84 to 96 w %, with the addition of benzalkonium chloride (less than 0.1 w %).

### Example 4: Pharmaceutical Composition Comprising Lopinavir

The composition comprises a micro-emulsion incorporating lopinavir at a concentration of from 1 to 50 mg/ml, wherein the micro-emulsion is made of triglycerides LABRAFAC®/Span 80/water with respective amount of 77 to 93 w %/5 to 15 w %/2 to 8 w %, with the addition benzalkonium chloride (less than 0.1 w %).

### Example 5: Pharmaceutical Composition Comprising Tenofovir Disoproxil Fumarate

The composition comprises a hot water-in-oil micro-emulsion incorporating tenofovir disoproxil fumarate (TDF) at a concentration of from 1 to 100 mg/ml preferentially 4 mg/ml, wherein the oil phase comprises Capmul® MCM EP-NF/Span 80/Polysorbate 80/Transcutol/Propylene glycol and water with respective amounts of 30/3/7/25/5/30 (in weight), with the addition of benzalkonium chloride (less than 0.1 w %)

The brain and plasma levels of (TDF) after the administration of the composition to mice at the dose of 4 mg/kg by the oral (n=6) or the nasal route (n=6) over 5 days have been measured and are shown in the Table below:

Dosage 4 mg/kg over 5 days

| Route of | | BRAIN | PLASMA |
|---|---|---|---|
| administration | | (ng/g) | (ng/ml) |
| ORAL | Mean | <1 | 25.3 |
| | SD | — | 33.9 |
| NASAL | Mean | 4.0 | 37.1 |
| | SD | 5.1 | 33.8 |

TDF concentrations in brain and plasma of mice (n = 6)

TDF is significantly (more than 4-fold) more concentrated in the brain after nasal administration than after oral administration. Plasma bioavailability is also higher (+47%) after nasal administration but proportionally less than for brain concentrations. This indicates that after the nasal administration with the composition according to the invention, TDF penetrates into the brain by the nose-to-brain pathway.

1. A method for preventing or treating an infection by a neurotropic virus in an individual, comprising administering a pharmaceutical composition comprising at least one antiviral agent as an active agent by the nasal route.

2. The method of claim 1, wherein the pharmaceutical composition is administered by the nasal route through the olfactory nerve pathway.

3. The method of claim 1, wherein the antiviral agent as a molecular weight below 10 000 g/mol.

4. The method of claim 1, wherein the antiviral agent has a log P value below 2.

5. The method of claim 1, wherein the antiviral agent is a substrate of an efflux transporter of the individual.

6. The method of claim 1, wherein the neurotropic virus is Japanese Encephalitis virus, Venezuelan Equine Encephalitis virus, California encephalitis virus; polio virus, coxsackie virus, echo virus, mumps virus, measles virus, influenza virus, rabies virus, varicella-zoster virus, Epstein-Barr virus, cytomegalovirus, HHV-6 virus, rubella virus, JC virus, a virus of the Retroviridae family, a virus of the Herpesviridae family, or a virus of the Coronaviridae family.

7. The method of claim 1, wherein the virus is the Human Immunodeficiency Virus (HIV).

8. The method of claim 1, wherein the virus is SARS-CoV, SARS-CoV-2, MERS-CoV, or mutants or variants thereof.

9. The method of claim 8, wherein the virus is SARS-CoV-2, or a mutant or variant thereof.

10. The method of claim 1, further comprising preventing or treating anosmia and/or dysgeusia associated with the viral infection.

11. The method of claim 1, further comprising preventing or treating encephalopathy associated with the viral infection, neuro-inflammation associated with the viral infection, a neurological disorder associated with the viral infection, or a neurocognitive disorder associated with the viral infection.

12. The method of claim 1, further comprising preventing or treating HIV-associated neurocognitive disorder (HAND).

13. The method of claim 1, wherein the pharmaceutical composition further comprises at least one pharmaceutically acceptable vehicle, carrier or excipient suitable for an administration through the nasal route.

14. The method of claim 1, wherein the pharmaceutical composition further comprises at least one tensio-active, emulsifier or co-surfactant agent.

15. The method of claim 1, wherein the pharmaceutical composition is in the form of a water-in-oil micro-emulsion.

16. The method of claim 1, wherein the virus is HIV-1 or HIV-2.
